(19) United States
(12) Patent Application Publication
(10) Pub. No.: US 2015/0342513 A1
(43) Pub. Date: Dec. 3, 2015

(54) System and Method for Analyzing Biomechanics

(71) Applicant: INDUSTRIAL TECHNOLOGY RESEARCH INSTITUTE, Hsinchu (TW)

(72) Inventors: Chi-Kang Wu, Taipei City (TW); Yu-Wen Chen, Tainan City (TW); Hian-Kun Tenn, Kaohsiung City (TW); Chih-Hung Huang, Xinying City (TW)

(21) Appl. No.: 14/822,952

(22) Filed: Aug. 11, 2015

Related U.S. Application Data
(63) Continuation-in-part of application No. 12/842,244, filed on Jul. 23, 2010.

(30) Foreign Application Priority Data
Jul. 1, 2010 (TW) 099121749

(51) Int. Cl. A61B 5/22; A61B 5/00; A61B 5/11

(52) U.S. Cl. CPC A61B 5/224; A61B 5/1107; A61B 5/725; A61B 5/1116; A61B 5/6828; A61B 5/7278; A61B 2562/0219

(57) ABSTRACT

A biomechanics analyzing system and a biomechanics computerized analyzing method for analyzing an organism when the organism performs an act by himself are provided. The biomechanics analyzing system includes an accelerometer, a low-pass filter, and a processing unit. The accelerometer is configured to be disposed on a surface of a muscle of the organism and is further configured to detect an acceleration signal. The low-pass filter is connected to the accelerometer and is configured for receiving the acceleration signal from the accelerometer and filtering the acceleration signal to produce a low-frequency signal. The processing unit is connected to the low-pass filter, and is configured for receiving the low-frequency signal from the low-pass filter and analyzing a frequency of a motion state of the organism according to the low-frequency signal.

This is a continuation-in-part application of application Ser. No. 12/842,244, filed on Jul. 23, 2010 which claims the benefit of Taiwan application Serial No.099121749, filed Jul. 1, 2010, the subject matter of which is incorporated herein by reference.

### TECHNICAL FIELD

The disclosure relates in general to a biomechanics analyzing system and a biomechanics analyzing method.

### BACKGROUND

Recently, the progress in the technology makes a lot of manpower be replaced with the mechanical power so that life becomes more convenient. However, the exercising opportunity of the human body is relatively gradually decreased. This causes the unbalanced enhancement in the physical fitness ability, and also degrades the training effect. The weakness of muscular fitness during exercise further causes frequently seen lifestyle diseases. For example, the low back pain is frequently caused by the muscular problem (i.e., the muscle weakness or muscle tightness) in the motion. At present, many references have proved that the enhancement of the strength of the muscle is advantageous to the maintenance of the health-related physical fitness of the non-athlete and the prevention of the modern lifestyle diseases.

### SUMMARY

According to an exemplary embodiment of the present disclosure, a biomechanics analyzing system for analyzing a motion state of an organism when the organism performs an act by himself is provided. The biomechanics analyzing system includes an accelerometer, a low-pass filter, and a processing unit. The accelerometer is configured to be disposed on a surface of a muscle of the organism and is further configured to detect an acceleration signal. The low-pass filter is connected to the accelerometer and is configured for receiving the acceleration signal from the accelerometer and filtering the acceleration signal to produce a low-frequency signal. The processing unit is connected to the low-pass filter, and is configured for receiving the low-frequency signal from the low-pass filter and analyzing a frequency of a motion state of the organism according to the low-frequency signal.

According to an exemplary embodiment of the present disclosure, a biomechanics computerized analyzing method for analyzing a motion state of an organism when the organism performs an act by himself is provided. The biomechanics computerized analyzing method includes the following steps. An acceleration signal is detected on a surface of a muscle of the organism by an accelerometer. The acceleration signal is filtered to produce a low-frequency signal. A frequency of the motion state of the organism is analyzed according to the low-frequency signal.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 is a block diagram showing a biomechanics analyzing system according to an embodiment of the disclosure.

FIGS. 2A to 3B are schematic illustrations showing a user wearing a detecting unit to do exercise.

FIG. 4 is a flow chart showing a biomechanics computerized analyzing method according to an embodiment of the disclosure.

FIG. 5 shows a low-frequency signal.

FIG. 6A shows a low-frequency signal.

FIG. 6B shows an acceleration signal.

FIG. 7 shows a relationship between the time and the median of the frequency of the motion state.

FIG. 8 shows a flowchart of detail steps for analyzing the muscle fatigue extent according to the frequency of the motion state.

FIG. 9 shows a flowchart of detail steps for analyzing the muscle endurance according to the muscle fatigue extent of the organism.

### DETAILED DESCRIPTION

The disclosure is directed to a biomechanics analyzing system and a biomechanics analyzing method for analyzing the mechanomyography (MMG) according to the acceleration signal detected by a detecting unit. Thus, the information, such as the posture and the frequency of the motion state of the user, can be obtained.

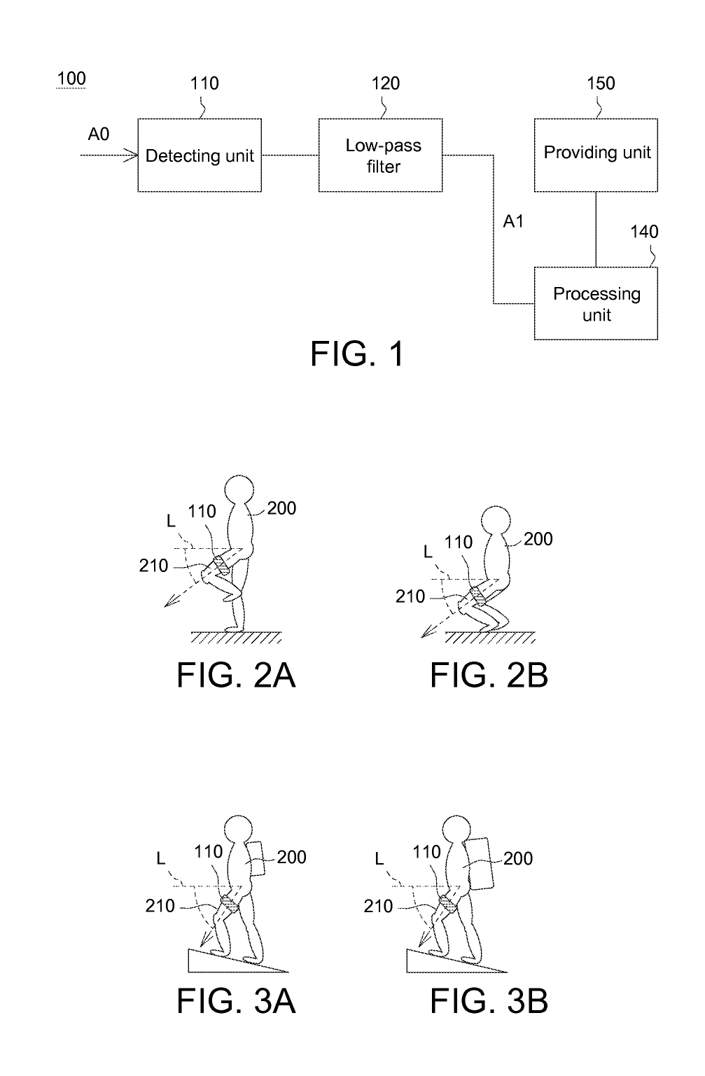

FIG. 1 is a block diagram showing a biomechanics analyzing system 100 according to an embodiment of the disclosure. Referring to FIG. 1, the biomechanics analyzing system 100 is for detecting a motion state of an organism. The organism may be an animal, such as the human, cat, dog, horse or fish. The biomechanics analyzing system 100 includes a detecting unit 110, a low-pass filter 120, a processing unit 140 and a providing unit 150. The detecting unit 110 detects an acceleration signal A0. For example, the detecting unit 10 may be a mechanical accelerometer, a piezoelectric voltage-type accelerometer, a charge-type accelerometer or a capacitive accelerometer. The low-pass filter 120 filters an electronic signal, and then lets the low-frequency components pass. The processing unit 140 analyzes various signals to obtain the associated information. The low-pass filter 120 and the processing unit 140 may be, for example, a chip, a firmware circuit or a computer readable recording medium for storing a plurality of sets of program codes. The providing unit 150, such as a hard disk, a memory card, a keyboard, a mouse or a transmission cable, provides a lot of required information.

FIGS. 2A to 3B are schematic illustrations showing a user 200 wearing the detecting unit 110 to do exercise. In FIG. 2A, the user 200 stands and lift his/her foot. The detecting unit 110 is worn on a thigh 210 of the user 200. The biomechanics analyzing system 100 (see FIG. 1) of this embodiment can analyze the angle of the thigh 210 with respect to the horizontal plane L to obtain the posture of the thigh 210 of the user 200. If the user 200 repeats the same motion, the biomechanics analyzing system 100 of this embodiment may also analyze its frequency of the motion state.

In FIG. 2B, the user 200 performs the semi-crouch motion. In FIGS. 2A and 2B, the angles of the thigh 210 with respect to the horizontal plane L are similar, but the strength of the muscle of the thigh 210 of FIG. 2B is greater than the strength of the muscle of the thigh 210 of FIG. 2A.

In FIG. 3A, the user 200 performs the hill climbing motion. The biomechanics analyzing system 100 (see FIG. 1) of this embodiment can analyze the angle of the thigh 210 with respect to the horizontal plane L to obtain the posture of the thigh 210 of the user 200. If the user 200 repeats the same motion, the biomechanics analyzing system 100 of this embodiment may also analyze its frequency of the motion state.

In FIG. 3B, the user 200 also performs the hill climbing motion, but the loading in FIG. 3B is greater than the loading in FIG. 3A, so that the strength of the muscle of the thigh 210 in FIG. 3B is greater than that in FIG.

3A.

Of course, in addition to the thigh 210, the detecting unit 110 may also be disposed on other extremities, the head, the breast, the waist, and the position thereof does not intend to restrict the disclosure.

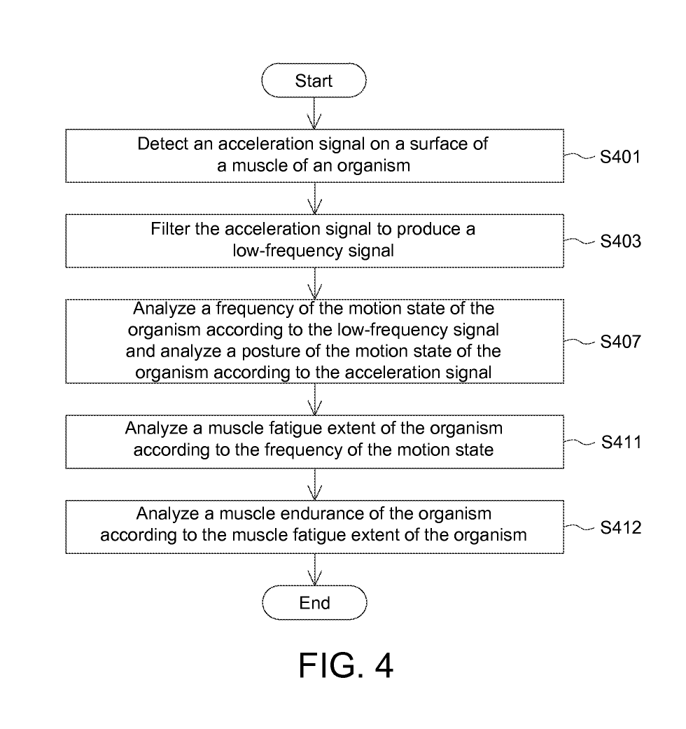

FIG. 4 is a flow chart showing a biomechanics computerized analyzing method according to an embodiment of the disclosure. As shown in FIGS. 1 and 4, the biomechanics computerized analyzing method of this embodiment will be clearly described with reference to an actual measurement example. In one actual measurement example, the detecting unit 110 is attached to the thigh of the user. Those skilled in the art may easily understand that the biomechanics analyzing system 100 of this embodiment is not particularly restricted to this flow chart, and the order and the contents of the steps may be properly adjusted.

First, in step S401, the detecting unit 110 is disposed on the surface of the muscle of the organism to detect an acceleration signal A0.

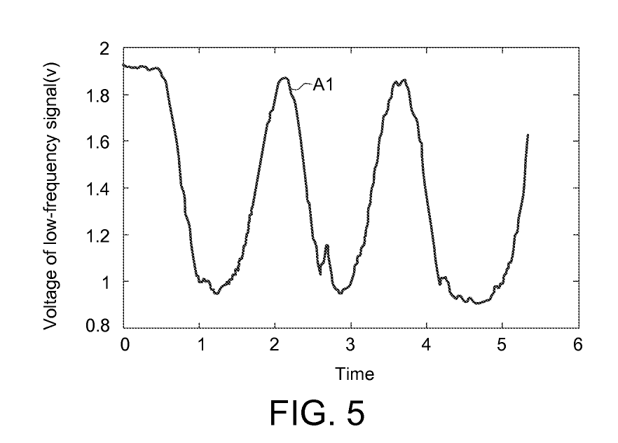

Next, in step S403, the low-pass filter 120 filters the acceleration signal A0 to produce a low-frequency signal A1. FIG. 5 shows the low-frequency signal A1.

Next, in step S407, the processing unit 140 analyzes a frequency of the motion state of the organism according to the low-frequency signal Al and analyzes a posture of the motion state of the organism according to the acceleration signal A0.

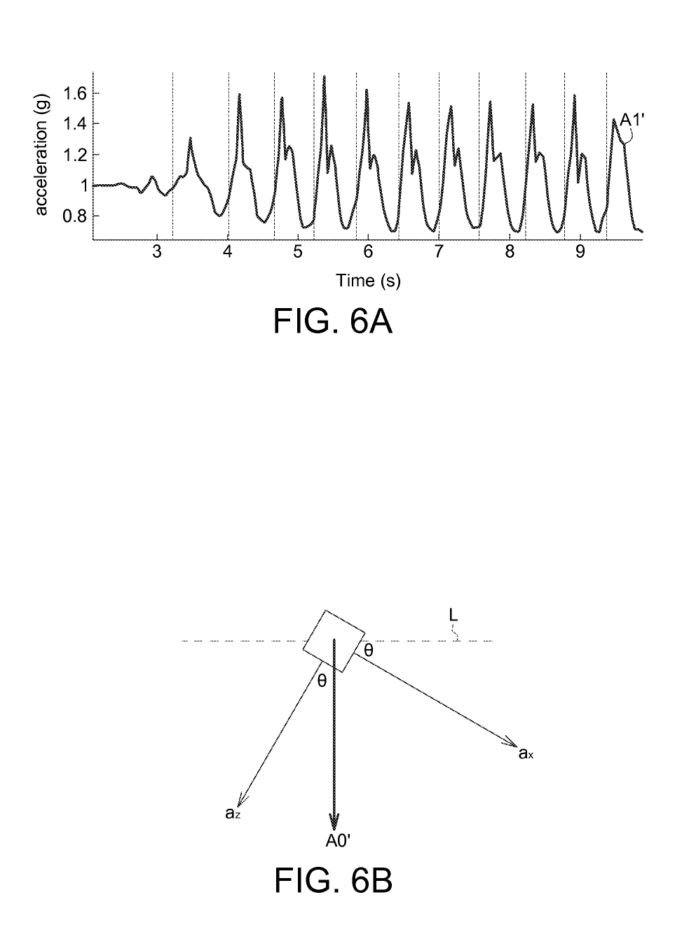

In one example, the frequency of the motion state of the organism can be analyzed according to the low-frequency signal A1 by the following steps. Please refer to FIG. 6A, which shows a low-frequency signal A1′. One organism wears an accelerometer when he is walking. The number of the local minimum of the low-frequency signal A1′ or the number of the local maximum of the low-frequency signal A1′ during a cycle time is deemed as the frequency of the walking steps.

In one example, the posture of the motion state of the organism can be analyzed according to an acceleration signal A0′ by the following steps.

Please refer to FIG. 6B, which shows the acceleration signal A0′. One organism wears the accelerometer on his thigh. The acceleration signal A0′ includes a X-axis acceleration aₓ and a Z-axis acceleration a_{z}. The angle θ with respect to the horizontal plane L can be calculated by θ=tan⁻¹(aₓ/a_{z}) Therefore, the angle of the thigh of the organism can be obtained. Then, the posture of the motion state of the organism can be obtained according to the angle of the thigh of the organism.

Next, in step S411, the processing unit 140 further analyzes a muscle fatigue extent of the organism according to the frequency of the motion state.

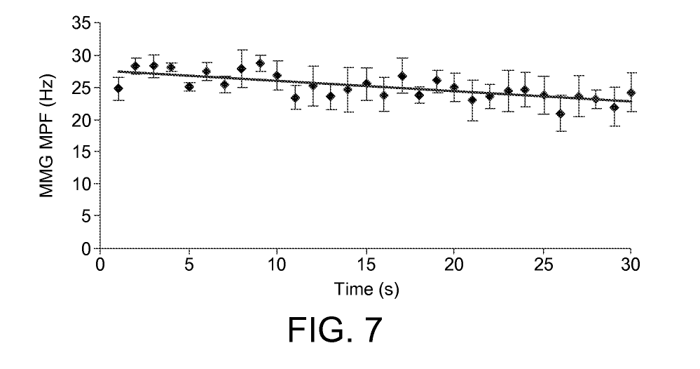

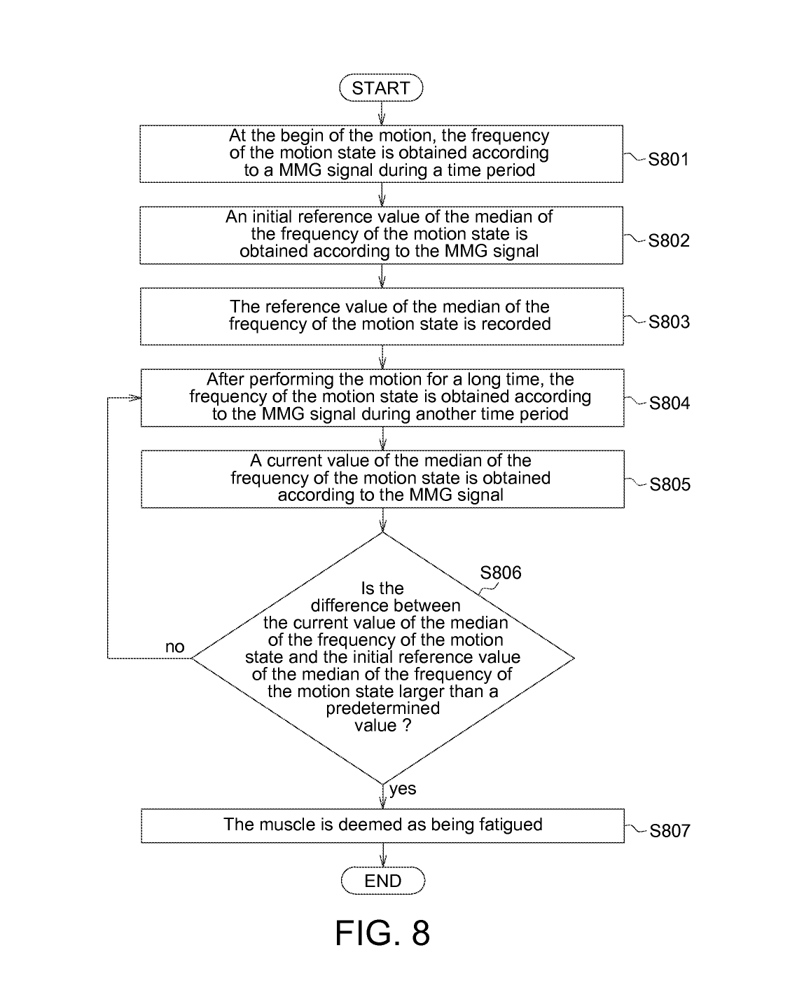

In one example, the muscle fatigue extent can be analyzed according to the frequency of the motion state by the following steps. Please refer to FIG. 7, which shows a relationship between the time and the median of the frequency of the motion state. Please refer to FIG. 8, which shows a flowchart of detail steps for analyzing the muscle fatigue extent according to the frequency of the motion state. Firstly, in step S801, at the begin of the motion, the frequency of the motion state is obtained according to a MMG signal during a time period. For example, the time period is 30 seconds. Then, in step S802, an initial reference value of the median of the frequency of the motion state is obtained according to the MMG signal. Next, in step S803, the initial reference value of the median of the frequency of the motion state is recorded. Then, in step S804, after performing the motion for a long time, the frequency of the motion state is obtained according to the MMG signal during another time period. Next, in step S805, a current value of the median of the frequency of the motion state is obtained according to the MMG signal. Then, in step S806, whether the difference between the current value of the median of the frequency of the motion state and the initial reference value of the median of the frequency of the motion state is larger than a predetermined value is determined. If the difference is larger than the predetermined value, then the process proceeds to the step S807. In step S807 the muscle is deemed as being fatigued.

Then, in step S412, the processing unit 140 further analyzes a muscle endurance of the organism according to the muscle fatigue extent of the organism.

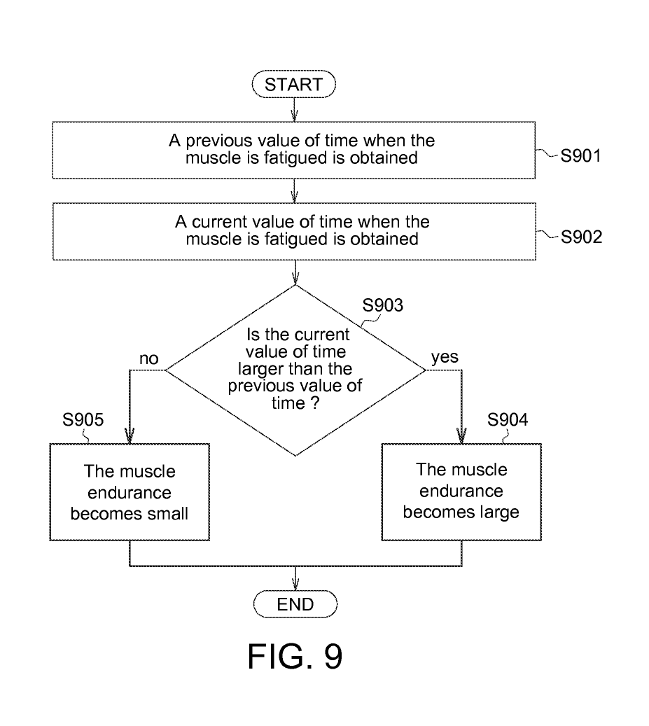

In one example, the muscle endurance can be analyzed according to the muscle fatigue extent of the organism by the following steps. Please refer to FIG. 9, which shows a flowchart of detail steps for analyzing the muscle endurance according to the muscle fatigue extent of the organism. Firstly, in step S901, a previous value of time when the muscle is fatigued is obtained. The previous value of time may be obtained before. Next, in step S902, a current value of time when the muscle is fatigued is obtained. Then, in step S903, whether the current value of time is larger than the previous value of time is determined. If the current value of time is larger than the previous value of time, then the process proceeds to step S904; otherwise, the process proceeds to step S905. In step S904, the muscle endurance becomes large. In step S905, the muscle endurance becomes small.

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A biomechanics analyzing system for analyzing an organism when the organism performs an act by himself, the system comprising:
an accelerometer configured to be disposed on a surface of a muscle of the organism and further configured to detect an acceleration signal;
a low-pass filter connected to the accelerometer, the low-pass filter configured for receiving the acceleration signal from the accelerometer and filtering the acceleration signal to produce a low-frequency signal; and
a processing unit connected to the low-pass filter, the processing unit configured for receiving the low-frequency signal from the low-pass filter, analyzing a frequency of a motion state of the organism according to the low-frequency signal.

2. The system according to claim 1, wherein the processing unit is further configured to analyze a posture of the motion state of the organism according to the acceleration signal.

3. The system according to claim 1, wherein the accelerometer is configured to be disposed on at least one extremity of the organism.

4. The system according to claim 1, wherein the accelerometer is a mechanical accelerometer, a piezoelectric voltage-type accelerometer, a charge-type accelerometer or a capacitive accelerometer.

5. The system according to claim 1, wherein the processing unit is further configured to analyze a muscle fatigue extent according to the frequency of the motion state.

6. The system according to claim 5, wherein the processing unit is further configured to analyze a muscle endurance of the organism according to the muscle fatigue extent of the organism.

7. A biomechanics computerized analyzing method for analyzing an organism when the organism performs an act by himself, the method comprising the steps of:
detecting an acceleration signal on a surface of a muscle of the organism by an accelerometer;
filtering the acceleration signal to produce a low-frequency signal; and
analyzing a frequency of a motion state of the organism according to the low-frequency signal.

8. The method according to claim 7, further comprising the step of:
analyzing a posture of the motion state of the organism according to the acceleration signal.

9. The method according to claim 7, further comprising the step of:
analyzing a muscle fatigue extent of the organism according to the frequency of the motion state.

10. The method according to claim 9, further comprising the step of:
analyzing a muscle endurance of the organism according to the muscle fatigue extent of the organism.
